(19) United States
(12) Patent Application Publication
(10) Pub. No.: US 2023/0364198 A1
(43) Pub. Date: Nov. 16, 2023

(54) C-Peptide as a Therapy for Patients with Diabetes

(71) Applicant: University of Cincinnati, Cincinnati, OH (US)

(72) Inventor: Jason J. Winnick, Cincinnati, OH (US)

(73) Assignee: University of Cincinnati, Cincinnati, OH (US)

(21) Appl. No.: 18/196,168

(22) Filed: May 11, 2023

Related U.S. Application Data
(60) Provisional application No. 63/340,723, filed on May 11, 2022.

(51) Int. Cl. A61K 38/28; A61K 38/26; A61K 38/31; A61P 3/10

(52) U.S. Cl. CPC A61K 38/28; A61K 38/26; A61K 38/31; A61P 3/10

(57) ABSTRACT

A method of reducing the incidence of hypoglycemia in an insulin-requiring subject is provided. The subject is administered insulin either via regular doses of insulin or via continuous subcutaneous infusion. The method involves administering to the subject C-peptide in conjunction with at least a portion of the administered insulin, where the insulin dosage is not reduced from the subject’s regular dosage as a result of the administration of C-peptide.

### CROSS-REFERENCE TO RELATED APPLICATIONS

This application claims priority to, and the benefit of the filing date of, U.S. Patent Application Serial No. 63/340,723, filed on May 11, 2022, the disclosure of which is incorporated by reference herein in its entirety.

### STATEMENT REGARDING FEDERALLY FUNDED RESEARCH OR DEVELOPMENT

This invention was made with government support under grant DK-106364, awarded by the National Institutes of Health, the National Institute of Diabetes and Digestive and Kidney Diseases. The U.S. Government has certain rights in the invention.

### TECHNICAL FIELD

The present invention relates to diabetic treatments.

### BACKGROUND OF THE INVENTION

Diabetes is a disease characterized by hyperglycemia. There are two types of diabetes. Type 1 diabetes occurs when an autoimmune response destroys the insulin-producing β-cells in the pancreas, thereby necessitating the administration of insulin subcutaneously. Type 2 diabetes is most often characterized by obesity and insulin resistance, and usually requires the use of insulin sensitizing or insulin potentiating medications. As type 2 diabetes progresses, many of these patients also require subcutaneous insulin.

Given that insulin-requiring patients estimate their own insulin needs during the fasting state or in response to the ingestion of glucose, the optimal dose is often overestimated. This results in hypoglycemia (low blood sugar), which can cause severe somatic symptoms. In response to a fall in blood glucose, counterregulatory hormones are secreted, including glucagon, epinephrine, cortisol and growth hormone, which act to counter insulin’s effect to lower blood glucose by enhancing glucose production by the liver and lipolysis by adipose tissue, and inhibiting glucose uptake by skeletal muscle. Unfortunately, the glucagon and epinephrine responses are usually impaired or lost in patients with diabetes, thereby making them even more susceptible to low blood sugar. Hypoglycemia leads to adverse health outcomes including hospitalization and death. It is among the most prominent obstacles to ideal blood glucose regulation. For this reason, it is a priority among the scientific community to discover ways to mitigate insulin-induced hypoglycemia, as this would dramatically improve patient care.

In healthy, non-diabetic individuals, insulin is stored in vesicles and released in response to a rise in blood glucose after eating a meal. However, insulin is not the only protein released by the β-cells; another protein, called C-peptide is secreted from these vesicles in equimolar doses with insulin. Of these two proteins, insulin’s pleiotropic effects on whole body glucose metabolism are well characterized, whereas despite numerous attempts to do so, no significant biological function of C-peptide has been identified to date. For this reason, patients with diabetes administer insulin to regulate their blood sugar, and C-peptide is not included in this formulation.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In one aspect of the present invention, a method of reducing the incidence of hypoglycemia in an insulin-requiring subject who is administered insulin either via regular doses of insulin or via continuous subcutaneous infusion is provided. The method involves administering to the subject C-peptide in conjunction with at least a portion of the administered insulin, where the insulin dosage is not reduced from the subject’s regular dosage as a result of the administration of C-peptide. In one embodiment, the subject is a type-1 diabetic. In another embodiment, the subject is a type-2 diabetic.

In one embodiment, the subject has a condition selected from the group consisting of cystic fibrosis related diabetes, hypoglycemia resulting from a gastric bypass, hypoglycemia resulting from a sulfonylurea treatment, hyperinsulinemia resulting from endocrine tumors, and endocrine insufficiency. In another embodiment, the C-peptide is part of a larger molecule. In one embodiment, the larger molecule is selected from the group consisting of insulin, glucagon, somatostatin, GLP-1 or -2, GIP, cortisol, steroids, incretins, GLP, GIP, proinsulin, growth hormone, and other counterregulatory or postprandial hormones. In another embodiment, the C-peptide is chelated to form a larger molecule. In one embodiment, the C-peptide is PEGylated to form a larger molecule.

In another embodiment, the C-peptide is administered to the subject by a delivery method selected from the group consisting of injecting simultaneously with insulin, injecting separately from insulin, administering with an insulin pump, orally in a capsule or pill comprising both insulin and C-peptide, orally in a capsule or pill only comprising C-peptide as an active ingredient, cutaneously in a lotion that also contains insulin, and cutaneously in a lotion without insulin.

In another aspect of the present invention, a method of reducing the incidence of hypoglycemia in an insulin-requiring subject who is administered insulin either via regular doses of insulin or via continuous subcutaneous infusion is provided. The method involves administering to the subject C-peptide in conjunction with at least a portion of the administered insulin, where the molar dose ratio of C-peptide to insulin is greater than 1:1. In one embodiment, the molar dose ratio of C-peptide to insulin is greater than about 1.5:1. In another embodiment, the molar dose ratio of C-peptide to insulin is greater than about 2:1. In one embodiment, the subject is a type-1 diabetic. In another embodiment, the subject is a type-2 diabetic. In another aspect of the present invention, a method of reducing the incidence of hypoglycemia in an insulin-requiring subject who takes regular doses of insulin is provided. The method involves administering to the subject a dose of C-peptide in conjunction with a dose of insulin, where the molar dose ratio of C-peptide to insulin is less than 1:1. In one embodiment, the molar dose ratio of C-peptide to insulin is less than about 0.75:1. In another embodiment, the molar dose ratio of C-peptide to insulin is less than about 0.5:1.

In one embodiment, the subject has a condition selected from the group consisting of cystic fibrosis related diabetes, hypoglycemia resulting from a gastric bypass, hypoglycemia resulting from a sulfonylurea treatment, hyperinsulinemia resulting from endocrine tumors, endocrine insufficiency, and a high blood sugar condition requiring an intravenous (IV) infusion of insulin. In another embodiment, the C-peptide is part of a larger molecule. In one embodiment, the C-peptide can be chelated to form a larger molecule. In another embodiment, the C-peptide can be PEGylated to form a larger molecule. In one embodiment, the larger molecule is selected from the group consisting of insulin, glucagon, somatostatin, GLP-1 or -2, GIP, cortisol, steroids, incretins, GLP, GIP, proinsulin, and other counterregulatory or postprandial hormones. In another embodiment, the C-peptide is administered to the subject by a delivery method selected from the group consisting of injecting simultaneously with insulin, injecting separately from insulin, administering with an insulin pump, orally in a capsule or pill comprising both insulin and C-peptide, orally in a capsule or pill only comprising C-peptide as an active ingredient, cutaneously in a lotion that also contains insulin, and cutaneously in a lotion without insulin.

In another aspect of the present invention, a method of reducing the incidence of hypoglycemia in an insulin-requiring subject who is administered insulin either via regular doses of insulin or via continuous subcutaneous infusion is provided. The method involves administering to the subject C-peptide when the subject is exercising or before the subject begins exercising. In one embodiment, the subject is a type-1 diabetic. In another embodiment, the subject is a type-2 diabetic. In one embodiment, the subject has a condition selected from the group consisting of cystic fibrosis related diabetes, hypoglycemia resulting from a gastric bypass, hypoglycemia resulting from a sulfonylurea treatment, hyperinsulinemia resulting from endocrine tumors, and endocrine insufficiency. In another aspect of the present invention, an ergogenic aid comprising C-peptide is provided. In one embodiment, the C-peptide ergogenic aid is administered at a level from about 250-5000 micrograms per day. In another embodiment, the C-peptide ergogenic aid is administered at a level from about 500-1000 micrograms per day.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention. Similar reference numerals are used to indicate similar features throughout the various figures of the drawings.

FIG. 1 is a schematic representation of the metabolic studies of the present invention.

FIG. 2A is a graph showing hormonal responses during the euglycemic (eugly) Pd1 and the hypoglycemic (hypo) Pd2 for arterial insulin. *P < 0.001 between treatments. Data were analyzed using 2-way repeated measures ANOVA. CPEP, C-peptide; Pd1, euglycemic period; Pd2, hypoglycemic period; SAL, saline.

FIG. 2B is a graph showing hormonal responses during the euglycemic (eugly) Pd1 and the hypoglycemic (hypo) Pd2 for hepatic sinusoidal insulin.

FIG. 2C is a graph showing hormonal responses during the euglycemic (eugly) Pd1 and the hypoglycemic (hypo) Pd2 for C-peptide.

FIG. 2D is a graph showing hormonal responses during the euglycemic (eugly) Pd1 and the hypoglycemic (hypo) Pd2 for epinephrine.

FIG. 2E is a graph showing hormonal responses during the euglycemic (eugly) Pd1 and the hypoglycemic (hypo) Pd2 for norepinephrine.

FIG. 2F is a graph showing hormonal responses during the euglycemic (eugly) Pd1 and the hypoglycemic (hypo) Pd2 for cortisol.

FIG. 3A is a graph showing glucagon responses during the euglycemic Pd1 and the hypoglycemic Pd2 for arterial plasma. *P ≤ 0.05 between treatments. #P ≤ 0.10 between treatments. Time course data were analyzed using 2-way repeated measures ANOVA. Data for Δ AUC were analyzed using paired 1-way t test.

FIG. 3B is a graph showing the Δ AUC values during the final 90 minutes of euglycemic Pd1 and hypoglycemic Pd2 for arterial plasma.

FIG. 3C is a graph showing glucagon responses during the euglycemic Pd1 and the hypoglycemic Pd2 for hepatic portal vein plasma.

FIG. 3D is a graph showing the Δ AUC values during the final 90 minutes of euglycemic Pd1 and hypoglycemic Pd2 for hepatic portal vein plasma.

FIG. 3E is a graph showing glucagon responses during the euglycemic Pd1 and the hypoglycemic Pd2 for hepatic sinusoidal plasma.

FIG. 3F is a graph showing the Δ AUC values during the final 90 minutes of euglycemic Pd1 and hypoglycemic Pd2 for hepatic sinusoidal plasma.

FIG. 4A is a graph showing glucoregulatory responses during the euglycemic Pd1 and the hypoglycemic Pd2 for arterial plasma glucose. *P ≤ 0.05 between treatments. #P = 0.06 between treatments. Time course data were analyzed using 2-way repeated measures ANOVA. AUC data were analyzed using paired 2-way t test.

FIG. 4B is a graph showing the exogenous glucose infusion rate (GIR) and the GIR AUC during the final 90 minutes of Pd2 (inset).

FIG. 4C is a graph showing the net hepatic glucose balance.

FIG. 4D is a graph showing the AUC for net hepatic glucose balance during the final 90 minutes of Pd2.

FIG. 5A is a graph showing glucagon secretory responses during the euglycemic Pd1 and during the hypoglycemic Pd2 for glucagon secretion. *P ≤ 0.05 between treatments. Time course data were analyzed using 2-way repeated measures ANOVA. Data for Δ AUC were analyzed using paired 2-way t test.

FIG. 5B is a graph showing the Δ AUC values (last 90 minutes) for glucagon secretion during Pd1.

FIG. 5C is a graph showing the Δ AUC values (last 90 minutes) for glucagon secretion during Pd2.

FIG. 6 is a table showing the total hepatic blood flow, non-hepatic glucose uptake, and the arterial concentrations and net hepatic balance of lactate, NEFA, and glycerol.

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, “a portion” means a single dose when doses of insulin are administered. “A portion” may also mean at least 1% of insulin administered daily using continuous subcutaneous infusion.

As used herein, “continuous subcutaneous infusion” means the administration of a medication such as insulin using an electromechanical pump that infuses the medication, typically at pre-selected rates. The rate can be boosted by the patient as required for food intake. The pump typically comprises a battery-operated motor, a computerized control mechanism, an insulin reservoir and an infusion set (subcutaneous cannula and tubing). Sensor-augmented pumps can also be used, in which the pump is integrated with a real-time continuous glucose monitor (CGM). Patch pumps (tubing-free pumps in which the reservoir and integrated infusion set adhere to the skin) may also be used.

As used herein, “ergogenic aid” means a mechanical, nutritional, pharmacological, physiological and/or psychological tool that athletes use to increase energy, performance and recovery.

As used herein “in conjunction” means either administered simultaneously or separately, but within a time period of a few minutes.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers’ specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

The present invention involves the co-administration of C-peptide with insulin to reduce the incidence of hypoglycemia in insulin-requiring patients, including some patients with diabetes. Type 1 diabetes (T1D) is an autoimmune disease in which most islet β cells are destroyed, leading to insulin deficiency and an inability to maintain blood glucose homeostasis. Since Banting, Macleod, and colleagues first treated T1D patients with insulin 100 years ago, the treatment of the disease has been transformed such that it is manageable with the self-administration of insulin. One of the negative side effects of insulin use, however, is that it can lead to dose overestimations and hypoglycemia. In fact, patient fear of hypoglycemia continues to be a prominent barrier to optimal glycemic control, thereby increasing the risk of developing macro- and microvascular complications. Further increasing the risk of iatrogenic hypoglycemia in T1D is that the counterregulatory hormone responses to hypoglycemia are severely diminished in many of these patients.

Under overnight fasting conditions in healthy adults, the liver produces glucose at a rate of approximately 2.2 mg/kg/min to sustain euglycemia (~90 mg/dL). This rate of production is equal to that of glucose utilization by the rest of the body, approximately 50% to 60% of which is accounted for by the brain. The precise maintenance of euglycemia is achieved by subtle, minute-to-minute changes in the secretion of glucagon and insulin, which act in opposition to one another to increase or decrease hepatic glucose production (HGP), respectively. A portion of the regulation of glucagon secretion has been ascribed to an inhibitory effect of insulin on glucagon secretion, also known as the intraislet hypothesis. In fact, the intravenous (IV) infusion of insulin during euglycemia has been shown to lower glucagon levels in dogs, healthy humans, and patients with diabetes. In contrast, endogenous insulin secretion, such as occurs after a meal, does not have the same inhibitory effect on glucagon levels, thereby raising the possibility that endogenous insulin regulates glucagon secretion differently than IV insulin.

In the event of insulin-induced hypoglycemia in nondiabetic people, formidable counterregulatory hormone responses exist to restore euglycemia. The first is a reduction in endogenous insulin secretion, followed by the release of glucagon and then an increase in adrenergic drive if the blood glucose level continues to fall. Of the latter 2 hormones, glucagon is known to have the higher glycemic threshold for its secretion, thereby making it the first line of defense against a fall in blood glucose, accounting for approximately 70% to 90% of HGP during the first 90 minutes of insulin-induced hypoglycemia. Most, but not all, studies have demonstrated that C-peptide positivity confers a protective effect against insulin-induced hypoglycemia. However, although this effect has been ascribed in the past to preserved insulin secretion, no studies have examined the impact of C-peptide per se on glucagon secretion in response to insulin-induced hypoglycemia. The present invention has found that C-peptide infusion will enhance glucagon secretion and HGP in response to insulin-induced hypoglycemia.

The examples presented below show that, in dogs, the intravenous infusion (IV) of C-peptide with insulin (compared to IV saline with insulin); 1) preserved glucagon secretion during euglycemic/ hyperinsulinemic conditions; and 2) doubled glucagon secretion during hypoglycemic/ hyperinsulinemic conditions. As a result, hepatic glucose production was increased under hypoglycemic conditions, which lowered the need for exogenous glucose to maintain glycemia at a set level of 50 mg/dL. These results support the inventive concept that the co-administration of C-peptide with insulin will reduce the incidence of hypoglycemia in insulin-requiring patients.

Previous publications have disclosed using C-peptide to improve the insulin sensitivity of insulin-requiring patients, which would mean that they require less insulin. This focused on the reduced need for insulin, and how failure to do so would result in hypoglycemia. In other words, that approach prevents hypoglycemia by lowering insulin delivery. The present invention, on the other hand, does not require insulin-requiring individuals who take C-peptide to lower their insulin doses. Instead, they can maintain the same dose or even increase it, because their ability to fight off low blood sugar would be improved.

In one embodiment, the present invention involves a method of reducing the incidence of hypoglycemia in an insulin-requiring subject who takes regular doses of insulin by administering to the subject a dose of C-peptide in conjunction with a dose of insulin, wherein the insulin dosage is not reduced from the subject’s regular dosage as a result of the administration of C-peptide. In another embodiment, the present invention involves a method of reducing the incidence of hypoglycemia in an insulin-requiring subject who takes regular doses of insulin by administering to the subject a dose of C-peptide in conjunction with a dose of insulin, wherein the molar dose ratio of C-peptide to insulin is greater than 1:1.

In various embodiments of the present invention, co-infusion of C-peptide with insulin can be used to reduce hypoglycemia in these populations: insulin requiring patients with type 1 diabetes; insulin requiring patients with type 2 diabetes; insulin requiring patients with cystic fibrosis related diabetes; gastric bypass patients who have hypoglycemia; sulfonylurea treatment; endocrine tumors that result in hyperinsulinemia; endocrine insufficiency; and other insulin-requiring patients including during sleep, when patients are most susceptible to hypoglycemia.

In another embodiment, the present invention involves the independent infusion of C-peptide in at least one or more of the following situations: during exercise in insulin requiring patients with type 1 diabetes; during exercise in insulin requiring patients with type 2 diabetes; during exercise in insulin requiring patients with cystic fibrosis related diabetes; or as an ergogenic aid in sports.

In another embodiment, the present invention involves administering greater than a 1:1 molar dose to prevent hypoglycemia. This may be recommended in any of the situations described above. In one embodiment, an approximate 1.5:1 molar dose of C-peptide to insulin is used for insulin-requiring diabetics. In another embodiment, an approximate 2:1 molar dose of C-peptide to insulin is used for insulin-requiring diabetics.

In another embodiment, the present invention involves the infusion of C-peptide that is part of a larger molecule (for example, bi- or tri-hormone, or more). Such larger molecules include: Insulin, Glucagon, Somatostatin, GLP-1 or -2, GIP, Cortisol (steroids), Incretins (GLP, GIP), Proinsulin, and other counterregulatory or postprandial hormones. In addition, the C-peptide can be chelated to form a larger molecule. For example, the C-peptide can be PEGylated to form a larger molecule.

The C-peptide of the present invention may be delivered in a variety of methods, including injecting with insulin, either simultaneously or separately; in conjunction with pump insulin, either simultaneously or separately, as an oral insulin/C-peptide delivery, either simultaneously or separately; cutaneously (using, for example, a lotion), either simultaneously or separately.

### Impact of C-Peptide on Insulin Treatment

T1D is a disease that continues to be effectively treated using subcutaneous insulin administration, allowing patients to lead a nearly normal life. Unfortunately, fear of hypoglycemia impedes optimal glycemic control, thereby making these patients more susceptible to developing macro- and microvascular complications. Patients who are C-peptide positive experience debilitating hypoglycemia less frequently than those without an insulinogenic reserve, although the impact of C-peptide per se on hypoglycemic counter-regulation has not been clearly defined. The data presented below indicates that in response to IV insulin infusion during euglycemia, the coinfusion of C-peptide prevented the expected decline in glucagon secretion. Moreover, C-peptide infusion during insulin-induced hypoglycemia doubled glucagon secretion and increased net hepatic glucose output by 75%, thereby lowering the need for exogenous glucose to maintain glycemia. These data make it evident that C-peptide can play a mitigating role in the suppression of glucagon secretion by insulin, which, if accessed, could lessen the risk of iatrogenic hypoglycemia in insulin-requiring individuals.

Postprandial levels of C-peptide depend on a number of factors, including meal composition and the presence of diabetes. The steady-state arterial value for the CPEP group was 17 ng/mL, which is higher than what is seen in dogs in the postprandial state (~2 ng/mL) but only somewhat higher than what is seen in humans (~10 ng/mL), a difference accounted for by a lower clearance rate of C-peptide in humans (~4.4 vs. ~11.6 mL/kg/min). On the other hand, the higher peripheral C-peptide levels generated in the examples were necessary to more closely mimic the postprandial level seen in the islets by glucagon-secreting α cells. Unlike insulin, C-peptide extraction by the liver is very small, only accounting for approximately 5% of its clearance, while most of the remaining fraction is accounted for by the kidneys, which argues against a direct effect of C-peptide on hepatic glucose metabolism during both Pd1 and Pd2 of our studies. In addition, the absence of any difference in peripheral glucose metabolism between treatments during Pd2, such as markers of lipolysis and non-hepatic glucose uptake, also supports the conclusion that the higher rates of net hepatic glucose output were a result of hyperglucagonemia and contributed to the diminished need for exogenous glucose.

There was a nonsignificant increase in adrenergic drive with C-peptide infusion, but the only hormone significantly impacted by C-peptide was glucagon. It has been reported that GPR-146 is the receptor for C-peptide and that these receptors are found in islet α cells, thereby making a stimulatory effect plausible. It is also known that in the absence of C-peptide, IV-delivered insulin lowers glucagon levels in vivo. Interestingly, the data presented below shows that C-peptide infusion during Pd1 (euglycemic period) reduced this suppressive effect on glucagon secretion, pointing toward a capability of preserving basal glucagon levels during hyper-insulinemia, which could be of benefit to insulin-requiring individuals between meals or while they sleep. Even more remarkable was that C-peptide’s effect on glucagon secretion was even more pronounced during hypoglycemia, leading to a 2-fold increase in glucagon secretion in the CPEP group compared with SAL, and a 75% increase in net HGP, which decreased the need for exogenous glucose infusion. Although the pathways contributing to increased HGP were not measured (i.e., gluconeogenesis and glycogenolysis), it is likely that enhanced glycogenolysis was the main driver because of glucagon’s known stimulatory impact on this process, especially during the first 90 minutes of insulin-induced hypoglycemia.

The finding of the present invention, that C-peptide infusion, alongside that of insulin, can enhance glucagon secretion and net hepatic glucose output when the blood sugar is low can have important clinical implications. For example, the coinfusion of C-peptide with insulin could lower the risk of complications in patients with T1D by allowing tighter glycemic control without an accompanying increase in the risk of hypoglycemia. In fact, this applies to all insulin-requiring individuals. While iatrogenic hypoglycemia is most closely associated with the pathology of T1D, it also impedes optimal glycemic regulation in insulin-requiring patients with other diseases, such as type 2 diabetes. In addition, after experiencing a bout of hypoglycemia, patients become even more susceptible to developing hypoglycemia because of further diminished counterregulatory hormone secretion, including glucagon. It is therefore possible that C-peptide coinfusion with insulin could make these people less vulnerable to low blood sugar in the wake of a previous hypoglycemic event.

In summary, our data demonstrate that IV-infused C-peptide preserves basal glucagon secretion during euglycemic-hyperinsulinemia in dogs, thereby suggesting that it plays a role in intraislet signaling and the in vivo regulation of glucagon secretion. Moreover, C-peptide infusion also doubled glucagon secretion in these animals during insulin-induced hypoglycemia, which increased net HGP by 75% and lowered the need for exogenous glucose infusion. These data show that C-peptide-positive T1D patients may be less susceptible to insulin-induced hypoglycemia because of enhanced counterregulatory responses conferred by C-peptide. The discoveries of the present invention could lead to novel therapies that lower the incidence of hypoglycemia and improve glycemic control in insulin-treated patients, thereby mitigating their risk of developing micro- and macrovascular complications.

### EXAMPLES

### Example 1: Basal Period

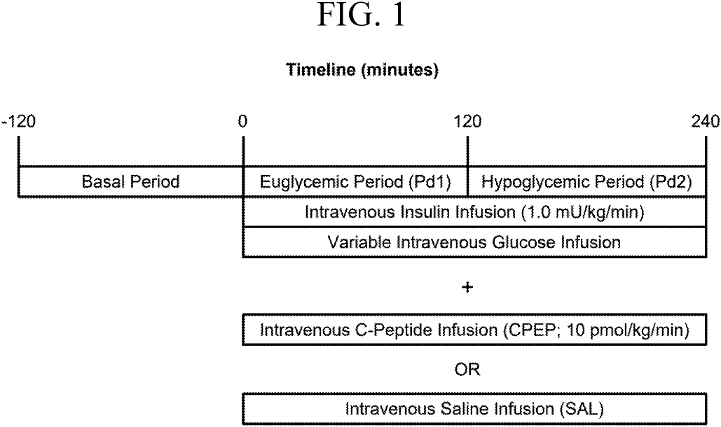

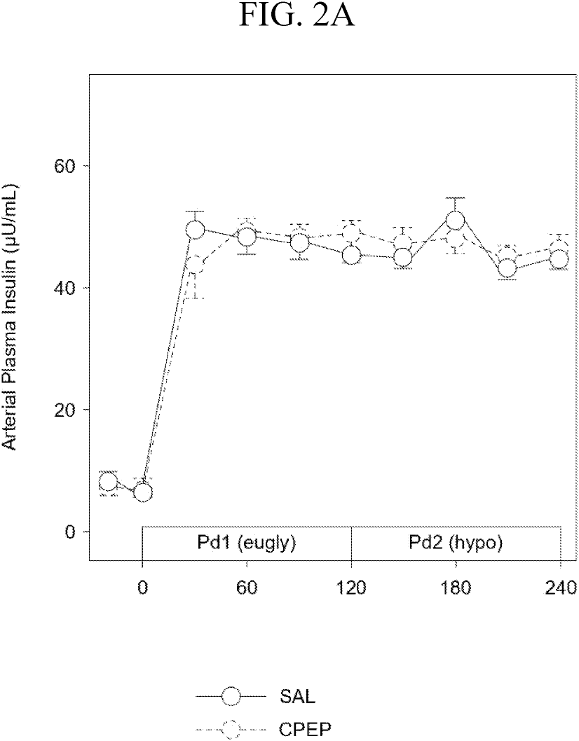

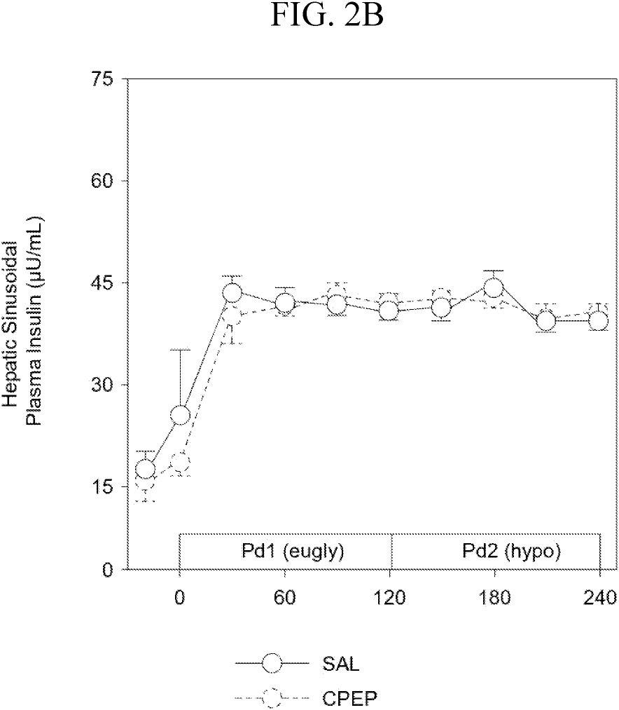

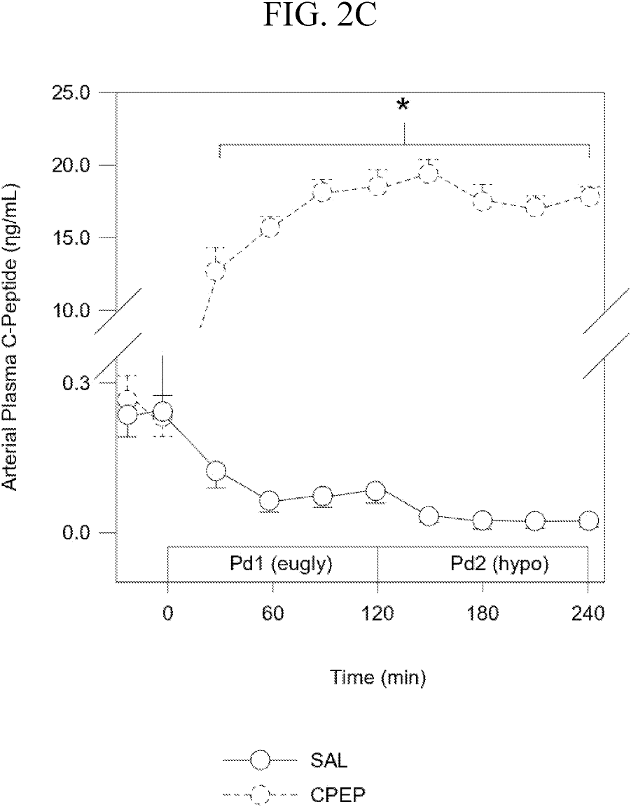

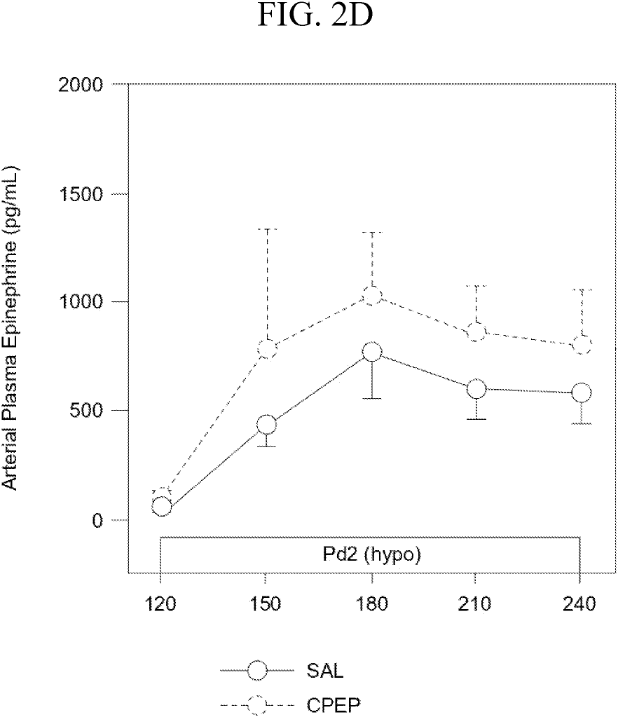

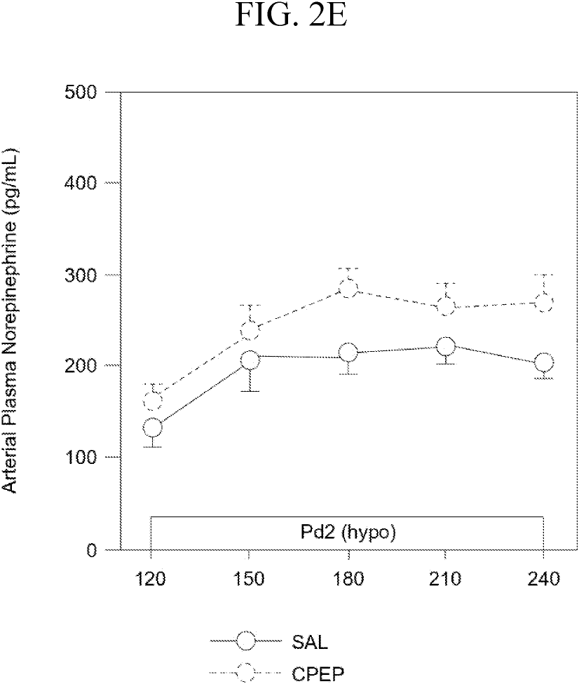

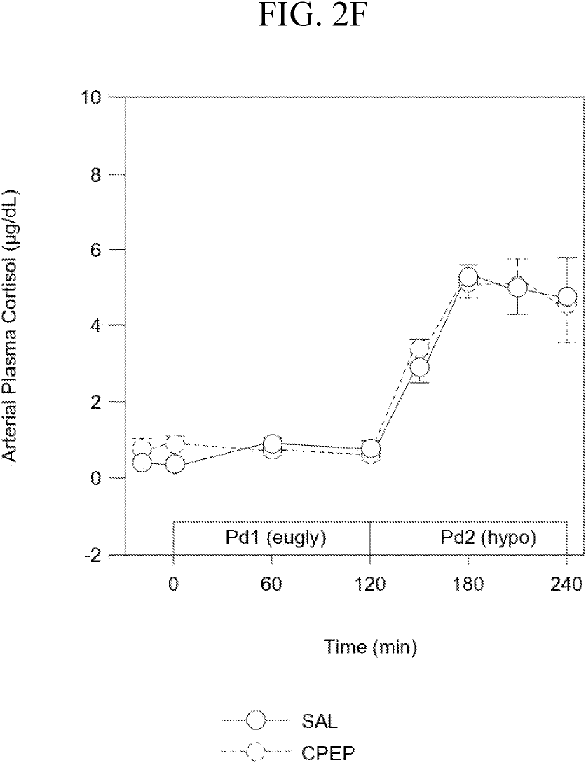

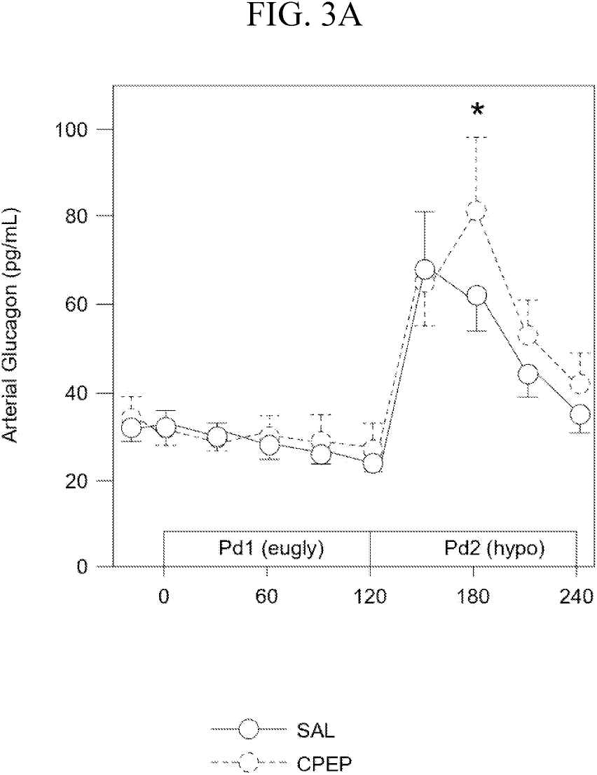

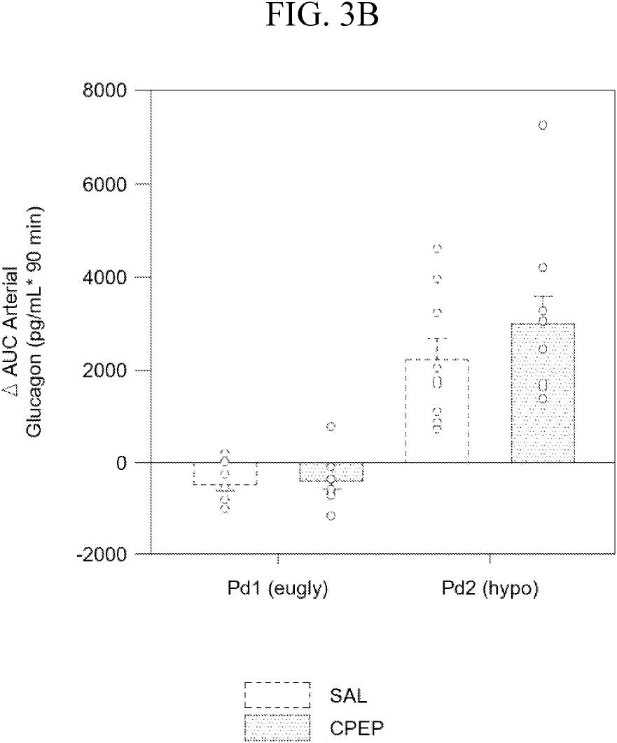

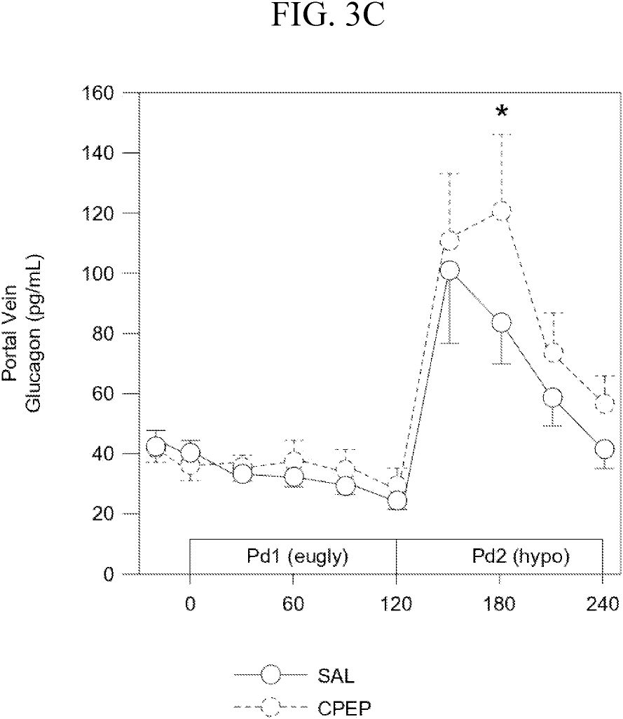

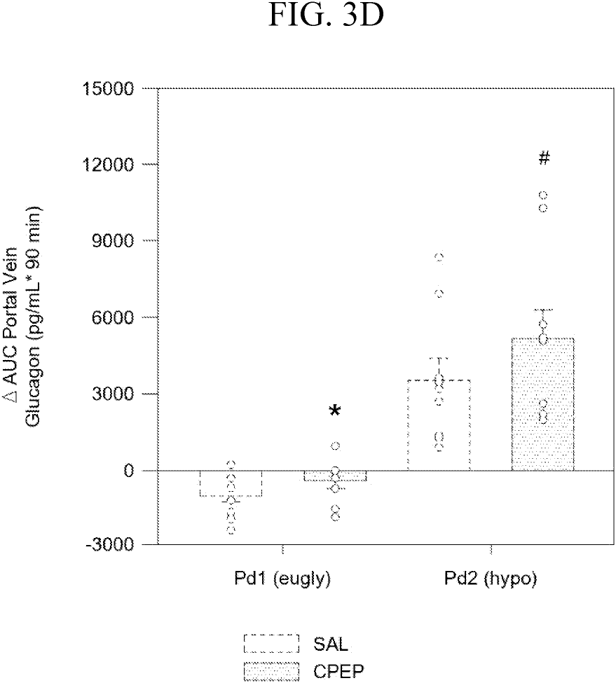

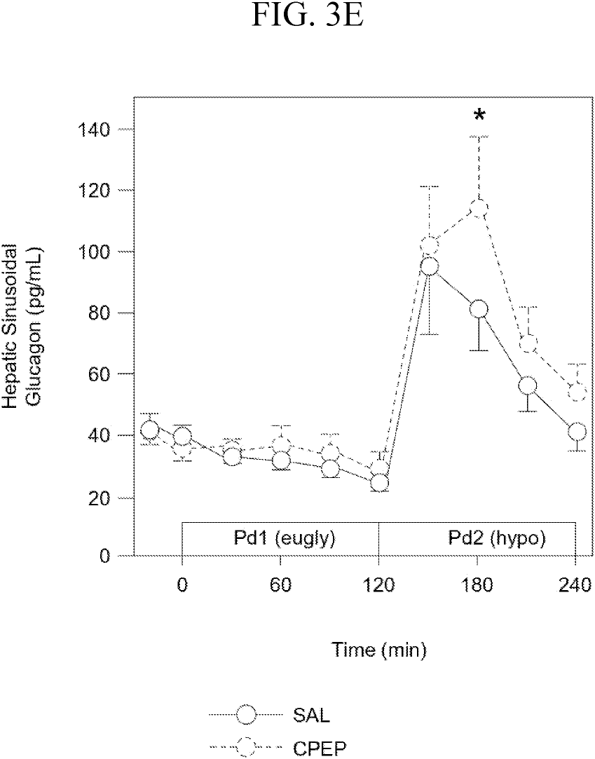

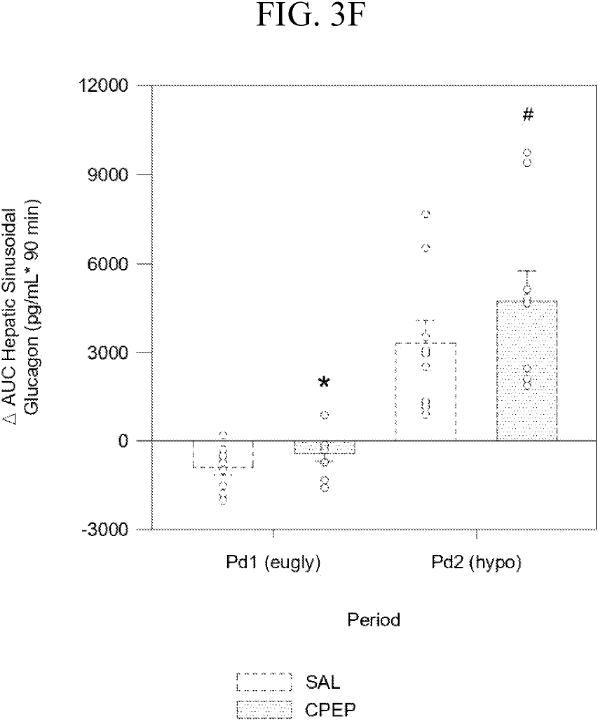

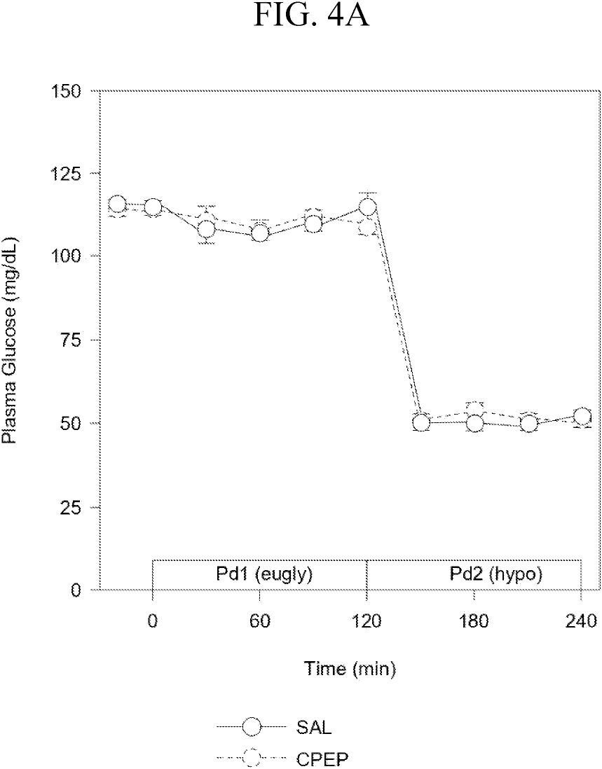

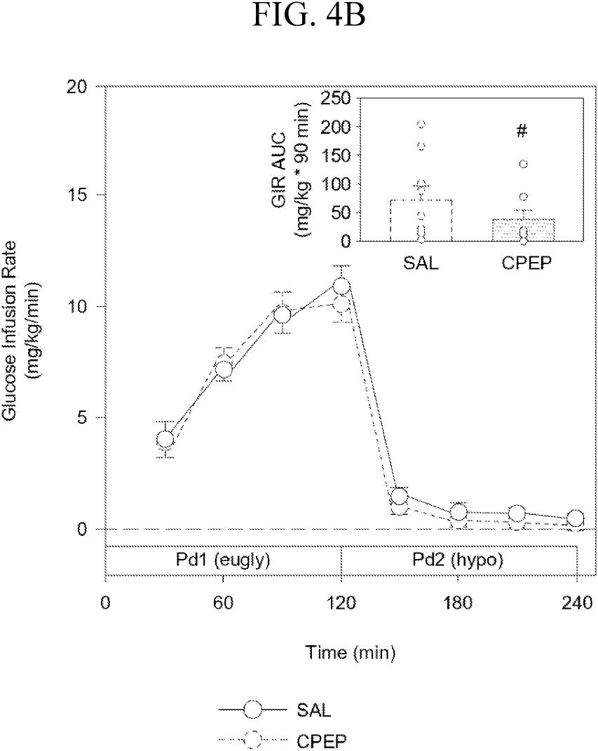

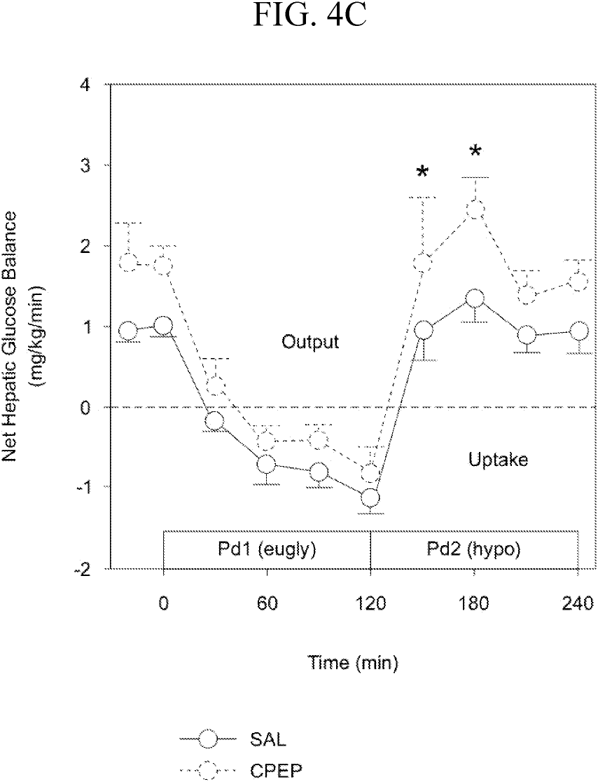

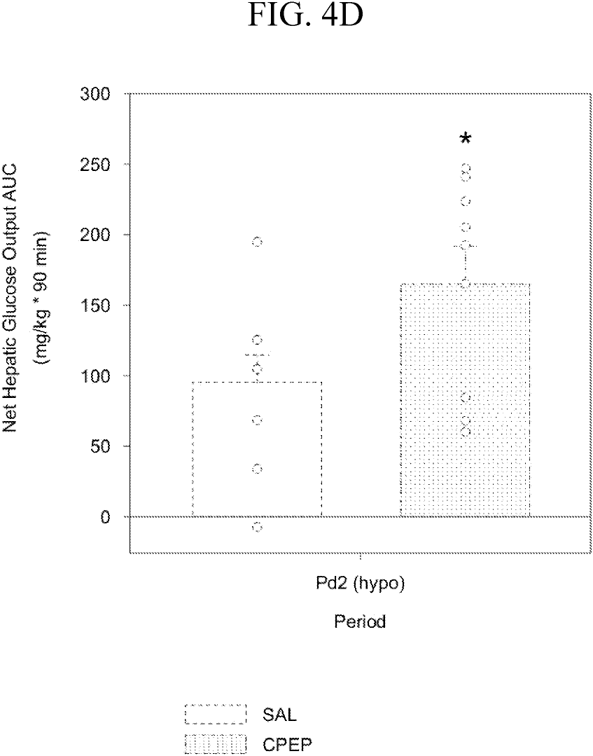

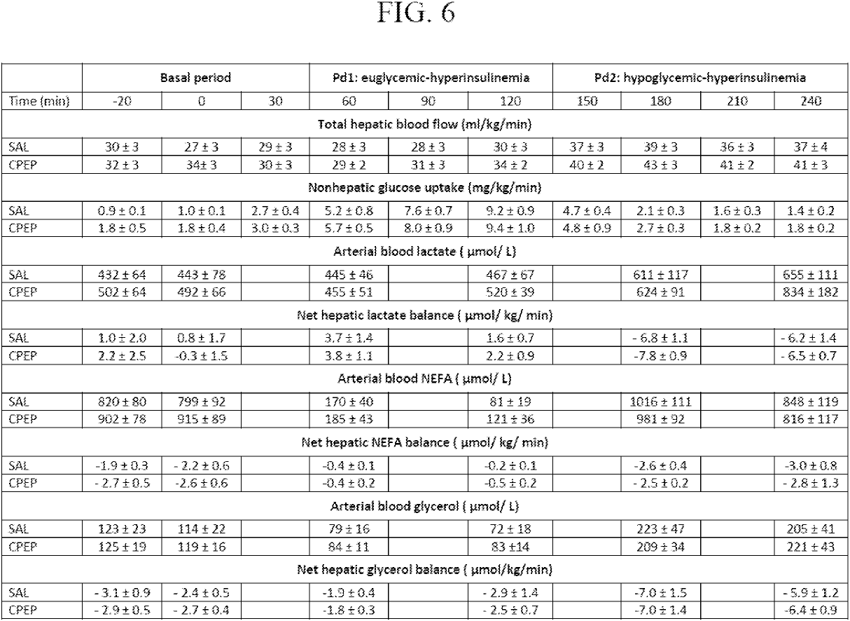

A study was conducted to determine if coinfusion of C-peptide and insulin would preserve basal glucagon secretion under euglycemic-hyperinsulinemic conditions. The design employed for this study is shown in FIG. 1. During the basal period (minute -20 to minute 0), plasma levels of insulin, cortisol, and C-peptide were similar between treatments (see FIGS. 2A-2F). Likewise, glucagon concentrations were similar in arterial, hepatic portal venous, and hepatic sinusoidal plasma (see FIGS. 3A-3F), and net hepatic glucose output and arterial plasma glucose levels were basal (see FIGS. 4A-4D). Hepatic blood flow, non-hepatic glucose uptake, and the metabolism of lactate, non-esterified fatty acids (NEFA), and glycerol were indistinguishable between treatment groups during the basal period (the table of FIG. 6).

### Example 2: Hyperinsulinemic/euglycemic Period Pd1

A Pd1 insulin infusion (±C-peptide, CPEP) led to a similar increase in insulin concentrations with both treatments (FIGS. 2A and 2B) and a marked increase in C-peptide in the CPEP condition (FIG. 2C). Neither hyperinsulinemia nor C-peptide impacted cortisol levels (FIG. 2F) or hepatic blood flow (the table of FIG. 6), and arterial glucose concentrations remained euglycemic (FIG. 4A) because of an IV infusion of dextrose that was indistinguishable between treatments (FIG. 4B).

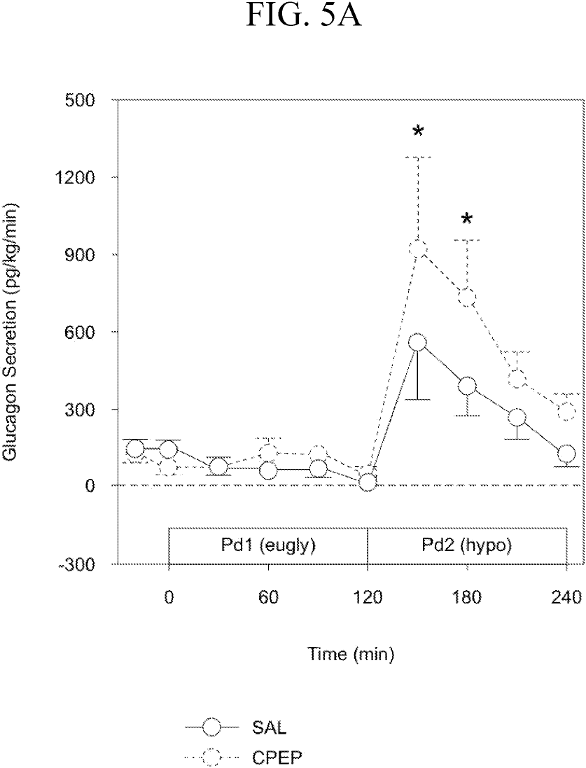

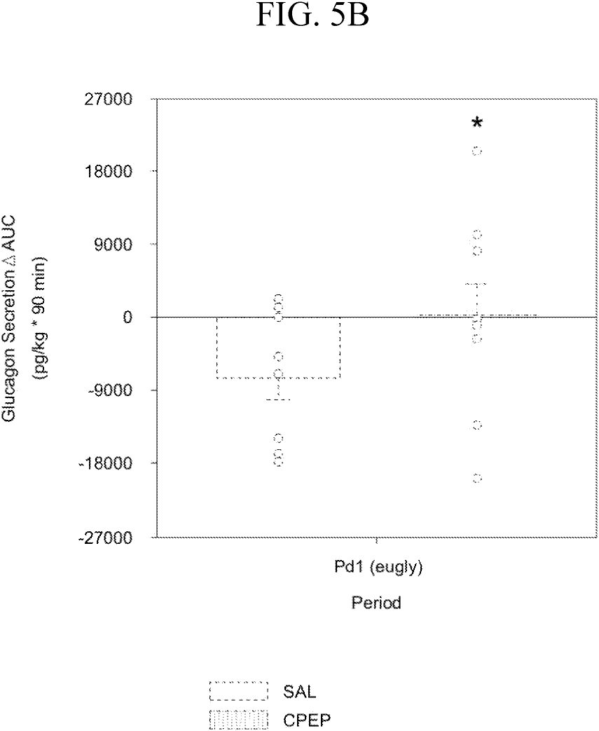

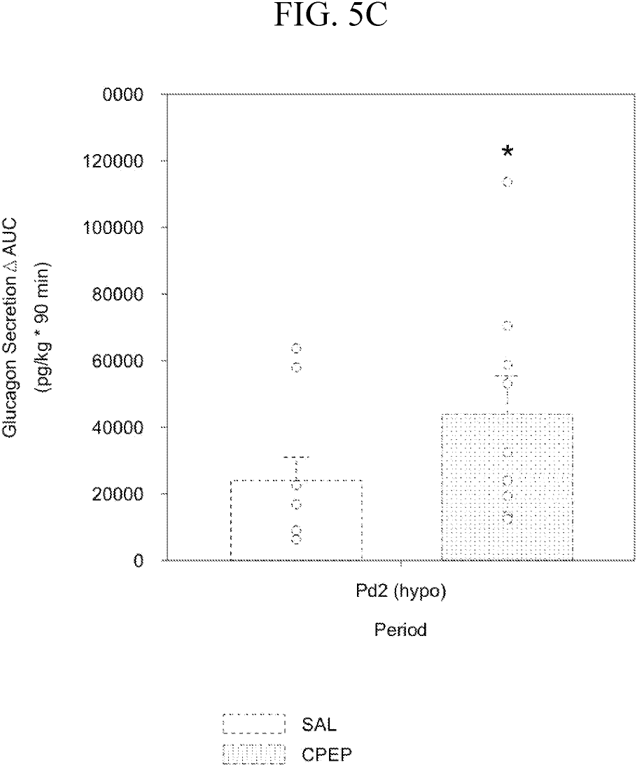

Arterial, hepatic portal venous, and hepatic sinusoidal glucagon concentrations were slightly higher in CPEP during the hyperinsulinemic/euglycemic conditions of Pd1, although these differences did not reach significance (FIGS. 3A, 3C, and 3E). On the other hand, the Δ AUC for portal venous and hepatic sinusoidal glucagon was less in CPEP compared with SAL, meaning that glucagon secretion decreased less in CPEP during Pd1 (FIGS. 3D and 3F). As expected, the greater reduction in glucagon in SAL was associated with a greater decrement in glucagon secretion (FIG. 5B). In response to hyperinsulinemia, net hepatic glucose output was completely suppressed in both treatments (FIG. 4C), in fact leading to slight net hepatic glucose uptake. Because of enhanced glucagon secretion in CPEP and the higher levels of glucagon in the hepatic sinusoids, net hepatic glucose uptake was somewhat lower in CPEP compared with SAL, although this did not reach statistical significance.

In response to the hyperinsulinemia of Pd1, lipolysis, as indicated by plasma NEFA and glycerol levels, was suppressed by approximately 90% in both treatments (the table of FIG. 6). As a result of diminished availability, net hepatic uptake of these substrates was reduced, with C-peptide having no additional impact (the table of FIG. 6). Arterial lactate levels were not impacted by hyperinsulinemia, nor were they altered by C-peptide infusion during Pd1 (the table of FIG. 6).

### Example 3: Hyperinsulinemic/Hypoglycemic Period Pd2

At the start of Pd2 (minute 120), the glucose infusion rate was reduced, and the plasma glucose level was allowed to fall to approximately 51 mg/dL (FIG. 4A). In response to insulin-induced hypoglycemia, epinephrine (FIG. 2D), norepinephrine (FIG. 2E), and cortisol (FIG. 2F) levels rose in SAL and CPEP, but there was no significant difference between treatments. As a result of enhanced glucagon secretion (FIGS. 5A and 5C), arterial, portal venous, and hepatic sinusoidal glucagon concentrations rose in both treatments (FIGS. 3A-3F), with the increase being greater with C-peptide infusion. In response to elevated glucagon levels at the liver in CPEP, net HGP during insulin-induced hypoglycemia was 73% higher in CPEP over the final 90 minutes of Pd2 (FIGS. 4C and 4D), leading to a 47% reduction in exogenous glucose required to sustain the plasma glucose at 50 mg/dL (AUC of 70 ± 25 vs. 37 ± 16 mg/kg × 90 minutes in SAL and CPEP, respectively; P = 0.06; FIG. 4B inset). In response to insulin-induced hypoglycemia, arterial lactate concentrations increased slightly, but similarly, in both treatments, as did net hepatic lactate uptake (the table of FIG. 6). Increased adrenergic drive to adipose tissue during hypoglycemia caused a sharp rise in NEFA and glycerol levels on each treatment day, which contributed to increased uptake of both by the liver (the table of FIG. 6), but C-peptide infusion had no impact on lipolysis.

### Example 4: Between Sex Analysis

A total of 5 male and 4 female dogs were studied. Statistical analysis did not reveal any between sex differences during Pd1 or Pd2 for glucagon secretion or net hepatic glucose output.

### METHODS

### Animal Care, Diet, Timeline, and Surgical Procedures

Studies were carried out on 18-hour fasted adult mongrel dogs (21 ± 4 kg; mean ± SD; 5 male, 4 female), aged 12.1 ± 0.6 months and acquired from a US Department of Agriculture-approved vendor. The animals were housed in a facility with a 12-hour light/12-hour dark cycle (lights on at 06:00 hours) and fed once daily a standard chow and meat diet (34% protein, 14.5% fat, 46% carbohydrate, and 5.5% fiber based on dry weight) that was weight maintaining.

Two weeks prior to being studied, each dog underwent surgery under general anesthesia to insert sampling catheters in a femoral artery, the hepatic portal vein, and the left common hepatic vein and to place blood flow probes (Transonic Systems) around the hepatic portal vein and hepatic artery. Two experiments were conducted on each animal, with the first being approximately 15 days after surgery and the second approximately 15 days later. Two days before each experiment, blood was drawn to measure leukocyte and hematocrit counts for each animal. Animals were only studied if they had a leukocyte count less than 16,000/mm³, a hematocrit more than 35%, a good appetite (noted by the consumption of at least 600 of the 800-calorie daily ration), and normal stools. On the morning of each experiment, the sampling catheters and flow probes were removed from their subcutaneous pockets under local anesthesia (2% lidocaine, Hospira), and the animal was placed in a Pavlov harness. Intravenous catheters were then inserted into a cephalic and saphenous vein to allow peripheral infusions as necessary. The animal was then allowed to rest for 100 minutes, after which basal samples were collected from the artery and portal and hepatic veins between -20 and 0 minutes.

After the 0-minute sample was collected, experimental period 1 (Pd1) began with the infusion of insulin at 1 mU/kg/min into a leg vein (FIG. 1). At the same time, a leg vein infusion of either canine CPEP (10 pmol/kg/min; AnaSpec) or SAL was also started, and dextrose was infused as needed to maintain euglycemia throughout Pd1 (0-120 minutes). Studies were randomized such that 4 of the 9 dogs received the C-peptide on the first study and the other 5 received saline first. At minute 120, the dextrose infusion was reduced, and the plasma glucose level was allowed to fall to approximately 50 mg/dL, where it was clamped (Pd2; 120-240 minutes; FIG. 1). At the end of the first experiment, all infusions were halted with the exception of glucose, which was infused as needed to restore euglycemia. Once that infusion was no longer required, the catheters and flow probes were placed back into subcutaneous pockets under general anesthesia. After the second experiment, animals were euthanized with pentobarbital, the abdomen was opened, and the positions of the catheter tips were verified.

### Specimen Analyses

The processing of blood samples has been described previously. Plasma glucose was analyzed using the glucose oxidase method (Analox Instruments). Insulin, glucagon, and C-peptide were measured using commercially available radioimmunoassay (RIA) kits from MilliporeSigma, and cortisol was measured using an RIA protocol developed by the Vanderbilt University Medical Center Hormone Assay & Analytical Services Core using an in-house antibody (gift from W. Nicholson) and 125I-cortisol (MP Biomedicals). Catecholamines were measured using high-performance liquid chromatography, while NEFA (FUJIFILM Medical Systems) and lactate and glycerol concentrations were measured using fluorometric assays.

### Calculations and Data Analysis

Hepatic blood flow was measured using ultrasonic flow probes (Transonic Systems). Net hepatic glucose balance (NHGB), hepatic sinusoidal insulin and glucagon levels, and non-hepatic glucose uptake were calculated as described previously, while plasma glucose levels were converted to whole blood values for the calculation of NHGB. Glucagon was measured every 30 minutes during Pd1 and Pd2, and those data are expressed as absolute values and as Δ AUC. The Δ AUC was calculated over the final 90 minutes of Pd1 and Pd2 as follows: ([AvgGGNt0-30 - Bs1GGN] × 30 min), where AvgGGNt0-30 refers to the average glucagon level over a given 30-minute period, Bs1GGN refers to the average glucagon level during the basal period (i.e., the average of the -20-minute and 0-minute time points), and 30 min represents the 30-minute sampling period. Three 30-minute calculations were then summed to provide the final number in units of pg/mL × 90 min. Glucagon secretion was calculated by multiplying the difference in plasma glucagon levels between the hepatic portal vein and artery by plasma flow in the hepatic portal vein.

### Statistics

All data are presented as mean ± SEM unless stated otherwise. Statistical analyses were carried out using SigmaStat software (Aspire Software International). Time course data were analyzed using 2-way ANOVA with repeated measures, and post hoc comparisons were made as appropriate. AUC data in FIGS. 3A-3F were analyzed using paired 1-way t test and in FIGS. 4A-4D and FIGS. 5A-5C were analyzed using paired 2-way t test. Significance was set at P < 0.05.

All documents cited are incorporated herein by reference; the citation of any document is not to be construed as an admission that it is prior art with respect to the present invention.

It is to be further understood that where descriptions of various embodiments use the term “comprising,” and / or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of reducing the incidence of hypoglycemia in an insulin-requiring subject who is administered insulin either via regular doses of insulin or via continuous subcutaneous infusion comprising administering to the subject C-peptide in conjunction with at least a portion of the administered insulin, wherein the insulin dosage is not reduced from the subject’s regular dosage as a result of the administration of C-peptide.

2. The method of claim 1 wherein the subject is a type-1 diabetic.

3. The method of claim 1 wherein the subject is a type-2 diabetic.

4. The method of claim 1 wherein the insulin is administered to the subject using continuous subcutaneous infusion.

5. The method of claim 1 wherein the subject has a condition selected from the group consisting of cystic fibrosis-related diabetes, hypoglycemia resulting from a gastric bypass, hypoglycemia resulting from a sulfonylurea treatment, hyperinsulinemia resulting from endocrine tumors, and endocrine insufficiency.

6. The method of claim 1 wherein the C-peptide is part of a larger molecule.

7. The method of claim 6 wherein the larger molecule is selected from the group consisting of insulin, glucagon, somatostatin, GLP-1 or -2, GIP, cortisol, steroids, incretins, GLP, GIP, proinsulin, and other counterregulatory or postprandial hormones.

8. The method of claim 1 wherein the C-peptide is administered to the subject by a delivery method selected from the group consisting of (i) injecting simultaneously with insulin, (ii) injecting separately from insulin, (iii) administering with an insulin pump, (iv) administering orally in a capsule or pill comprising both insulin and C-peptide, administering orally in a capsule or pill only comprising C-peptide as an active ingredient, administering cutaneously in a lotion that also contains insulin, and administering cutaneously in a lotion without insulin.

9. A method of reducing the incidence of hypoglycemia in an insulin-requiring subject who is administered insulin either via regular doses of insulin or via continuous subcutaneous infusion comprising administering to the subject C-peptide in conjunction with at least a portion of the administered insulin, wherein the molar dose ratio of C-peptide to insulin is greater than 1:1.

10. The method of claim 9 wherein the molar dose ratio of C-peptide to insulin is greater than about 1.5:1.

11. The method of claim 9 wherein the molar dose ratio of C-peptide to insulin is greater than about 2:1.

12. The method of claim 9 wherein the subject is a type-1 diabetic.

13. The method of claim 9 wherein the subject is a type-2 diabetic.

14. The method of claim 9 wherein the subject has a condition selected from the group consisting of cystic fibrosis related diabetes, hypoglycemia resulting from a gastric bypass, hypoglycemia resulting from a sulfonylurea treatment, hyperinsulinemia resulting from endocrine tumors, endocrine insufficiency, and a high blood sugar condition requiring an intravenous (IV) infusion of insulin.

15. The method of claim 9 wherein the C-peptide is part of a larger molecule.

16. The method of claim 15 wherein the larger molecule is selected from the group consisting of insulin, glucagon, somatostatin, GLP-1 or -2, GIP, cortisol, steroids, incretins, GLP, GIP, proinsulin, and other counterregulatory or postprandial hormones.

17. The method of claim 9 wherein the C-peptide is administered to the subject by a delivery method selected from the group consisting of injecting simultaneously with insulin, injecting separately from insulin, administering with an insulin pump, administering orally in a capsule or pill comprising both insulin and C-peptide, administering orally in a capsule or pill only comprising C-peptide as an active ingredient, administering cutaneously in a lotion that also contains insulin, and administering cutaneously in a lotion without insulin.

18. A method of reducing the incidence of hypoglycemia in an insulin-requiring subject who is administered insulin either via regular doses of insulin or via continuous subcutaneous infusion comprising administering to the subject C-peptide when the subject is exercising or before the subject begins exercising.

19. The method of claim 18 wherein the subject is a type-1 diabetic.

20. The method of claim 18 wherein the subject is a type-2 diabetic.

21. The method of claim 18 wherein the subject has a condition selected from the group consisting of cystic fibrosis-related diabetes, hypoglycemia resulting from a gastric bypass, hypoglycemia resulting from a sulfonylurea treatment, hyperinsulinemia resulting from endocrine tumors, and endocrine insufficiency.

22. An ergogenic aid comprising C-peptide.
